(19) United States
(12) Patent Application Publication
(10) Pub. No.: US 2005/0095288 A1
(43) Pub. Date: May 5, 2005

(54) Decongestant and expectorant tablets

(75) Inventor: Dickey Honea, Madison, MS (US)

Correspondence Address: HEDMAN & COSTIGAN P.C., 1185 AVENUE OF THE AMERICAS, NEW YORK, NY, 10036, US

(73) Assignee: Andrx Labs, LLC

(21) Appl. No.: 10/970,564

(22) Filed: Oct. 21, 2004

Related U.S. Application Data
(60) Provisional application No. 60/516,983, filed on Nov. 3, 2003.

(51) Int. Cl. A61K 31/075; A61K 9/20; A61K 9/22

(52) U.S. Cl. 424/464; 424/468; 514/718

(57) ABSTRACT

The present invention relates to a sustained release oral pharmaceutical tablet formulation containing an expectorant and a decongestant.

### CROSS REFERENCE TO RELATED APPLICATIONS

This application claims priority to U.S. Provisional Patent Application Ser. No. 60/516,983 filed Nov. 3, 2003.

### FIELD OF THE INVENTION

The present invention relates to the field of oral dosage forms and in particular to oral dosage tablet formulations that provide an extended release of an expectorant and a decongestant and an immediate release of an expectorant and/or decongestant.

### BACKGROUND OF THE INVENTION

The present invention relates to a twelve to twenty four hour unit dosage form comprising an expectorant and a decongestant. The unit dosage form is useful in treating patients for relief of nasal congestion due to the common cold, hay fever or other upper respiratory allergies, and nasal congestion associated with sinusitis; to promote nasal or sinus drainage; for the symptomatic relief of respiratory conditions characterized by dry nonproductive cough and in the presence of tenacious mucous and or mucous plugs in the respiratory tract.

One preferred expectorant in the present invention is guaifenesin, which is known chemically as 3-(2-methoxyphenoxy)-1,2-propane-diol. Guaifenesin is a drug that increases respiratory tract fluid secretions and helps to loosen phlegm and bronchial secretions. Guaifenesin increases the efficiency of a cough reflex and of ciliary action in removing accumulated secretions from trachea and bronchi by reducing the viscosity of secretions. Guaifenesin is readily absorbed from the intestinal tract and is rapidly metabolized and excreted in urine. Guaifenesin has a typical plasma half-life of approximately one hour. The currently known immediate release dosage tablets of guaifenesin provide only a short window of therapeutic effectiveness for patients due to the rapid metabolization and excretion of guaifenesin. This is a deficiency that the present invention seeks to alleviate.

Some common combinations of expectorants and decongestants are guaifenesin and phenylephrine sold commercially under the tradenames DECIBSAL®, SPRINKLE®, ENDAL® AND SINUPAN®, another known combination is guaifenesin and pseudoephedrine sold commercially under the tradenames CONGESTAC®, DECONSOL® and SINUFED®.

Phenylephrine, a decongestant, is known chemically as (αR)-3-hydroxy-α-[(methylamino)methyl]benzenemethanol. Phenylephrine is considered a potent pure alpha agonist drug that increases venous as well as arterial constriction. Phenylephrine has been found to provide excellent decongestion of the nasal mucosa by exerting its α-1 mediated vasoconstricting effect on the mucosal blood vessels. This directly opposes the histamine-mediated vasodilation and reduces mucosal edema and vascularity. It is commonly administered as phenylephrine hdrochloride.

Pseudoephedrine, a decongestant, is known chemically as (αS)-α-[(1S)-1-(methyamino)ethyl]benzenemethanol. Pseudoephedrine, as well as pharmaceutically acceptable acid addition salts thereof such as the hydrochloride and sulfate salts, is a sympathomimetic drug known by those skilled in the art as a safe therapeutic agent for treating nasal congestion. It is commonly administered orally and concomitantly with an antihistamine for treatment of nasal congestion for the treatment of allergic rhinitis. It is commonly administered as pseudoephedrine hdrochloride.

Twice daily controlled release formulations containing a combination of guaifenesin and phenylephrine or pseudoephedrine have been described. U.S. Pat. No. 6,462,064 describes compositions consisting of phenylephrine tannate and guaifenesin, and United States Patent Application No. 20030049318 describes sustained release formulations of guaifenesin and additional drug ingredients. This patent and application are incorporated herein by reference. The formulations described in this patent and application can be undesirable for many reasons including deficiencies in safety, effectiveness and ease of manufacture.

In order to encourage high patient compliance the expectorant/decongestant dosage unit should be prepared in a unit dosage form that is taken once or twice daily. These formulations should be stable, economical and easy to manufacture. These formulations should also be small enough for the patient to easily swallow.

It is therefore an objective of the present invention to provide a safe and effective twelve to twenty four hour expectorant and decongestant tablet formulation.

It is an objective of the present invention to provide a safe and effective twelve to twenty four hour dosage tablet formulation containing both an expectorant and a decongestant in which the tablet core is a single layer or homogeneous core that can be prepared by simple tabletting techniques such as direct compression and is therefore easy to manufacture.

### SUMMARY OF THE INVENTION

The present invention is an oral dosage tablet formulation that provides release of the active ingredients for up to twelve hours.

The core of the tablet can be a matrix, a controlled release coated core or an osmotic tablet.

The final tablet dosage form should provide an immediate release amount of the expectorant and/or the decongestant. For example if the tablet core is a matrix or osmotic tablet, a seal coat may optionally be applied to the tablet core followed by the application of an immediate release expectorant coating. The expectorant coating may then optionally be seal coated and a second immediate release coating comprising a decongestant may be applied to the seal coat. The decongestant coating also may be applied directly to the expectorant coating if a seal coating is not employed. The order in which the two immediate release coatings are applied can be reversed or the expectorant and decongestant may be combined into a single immediate release coating. Depending upon how the core is formed an immediate release decongestant or expectorant coating may not be needed. For example the matrix tablet may be formulated in a manner that allows for a therapeutic amount of expectorant or decongestant to be released in less than 2 hours, preferably less than 1 hour.

The final tablet dosage formulation may be optionally coated with a final seal coating and/or a polishing agent.

As used herein the terms “immediate release coating” or “immediate release amount” means a therapeutic amount of the expectorant and/or decongestant is released from the final dosage formulation within 2 hours, preferably within 90 minutes or less, and most preferably within 60 minutes or less when the dosage form is placed in 900 ml of simulated intestinal fluid, 37° C. and tested using a United States Pharmacopoeia Type II apparatus at 75 rpms.

Preferred embodiments of the present invention will comprise either 600 mg of guaifenesin and 15 mg of phenylephrine, 800 mg of guaifenesin and 20 mg of phenylephrine or 1200 mg of guaifenesin and 30 mg of phenylephrine. Additional preferred embodiments of the present invention will comprise either 600 mg of guaifenesin and 60 mg of pseudoephedrine, 800 mg of guaifenesin and 80 mg of pseudoephedrine or 1200 mg of guaifenesin and 120 mg of pseudoephedrine. The immediate release portions of the present invention should comprise about 10-65% of the total amount of the active ingredient(s) in the final dosage formulation, preferably about 15-55% of the total amount of the active ingredient(s) in the final dosage formulation and most preferably about 20-50% of the active ingredient(s) in the final dosage formulation. For example in the preferred embodiments described above the immediate release amount of guaifenesin will comprise about 200 mg, 265 mg and 400 mg respectively of the total guaifenesin in the final dosage formulation.

### DETAILED DESCRIPTION OF THE INVENTION

A preferred expectorant is guaifenesin, other possible expectorant include, but are not limited to, iodine products, terpinhydrate, ammonium chloride, beechwood creosote, potassium, guaiacolsufonate, syrup ipecac as well as pharmacologically acceptable salts thereof. A number of other expectorants are identified in Remington's 18^{th} Edition and incorporated herein by reference.

Preferred decongestants are phenylephrine and pseudoephedrine. Other possible decongestants include, but are not limited to, phenylpropanolamine, ephedrine, epinephrine, naphazoline, xylometazoline, oxymetazoline and phenylpropanolamine and other sympathomimetic drugs as well as pharmacologically acceptable salts thereof. A number of other decongestants are identified in Remington's 18^{th} Edition pp. 870-888, and are incorporated herein by reference.

The term “pharmacologically acceptable salts” encompasses both organic and inorganic acid addition salts including, for example those prepared from acids such as, sodium, hydrochloric, hydrofluoric, sulfuric, sulfonic, tartic, fumaric, hydrobromic, glycolic, citric, maleic, sulfate, phosphoric, succinic, acetic, nitric, benzoic, ascorbic, p-toluene sulfonic, benzenesulfonic, naphthalenesulfonic, propionic, and the like. Embodiments wherein the decongestant is pseudoephedrine as opposed to phenylephrine will comprise between 2 and 10 times the amount of decongestant per dosage unit, and preferably between 3 and five times the decongestant per dosage unit.

The tablet core can be any type of controlled release tablet core provided it is easily manufactured, controls the release of the active ingredients to provide therapeutic levels of the active ingredients for at least 12 hours after administration and is small enough to be easily swallowed. The tablet core can be a matrix, a controlled release coated core or an osmotic tablet.

Matrix Tablet Core

If the tablet core is a matrix, it should comprise the expectorant, the decongestant and a matrix forming agent which can be a wax, a hydrogel forming material such as a hydrogel forming polymer or a combination of the two. The matrix core will control the release of the expectorant and decongestant by a combination of diffusion of the active ingredients from the matrix and erosion of the matrix. The amount of diffusion and erosion will depend upon the materials selected for the formation of the matrix.

In one embodiment the matrix core is prepared by blending the following materials:

TABLE 1

| INGREDIENTS | PREFERRED | MOST PREFERRED |
|---|---|---|
| Expectorant | 10-80% | 20-70% |
| Decongestant | 10-50% | 20-40% |
| Hydrogel Forming polymer | 50-90% | 55-75% |
| Filler | 0-20% | 5-15% |
| Antiadherent | 0-5% | 0.01-2% |
| Lubricant (s) | 0-10% | 0.01-5% |

The above percentages are based on the total weight of the matrix core. Once the ingredients are blended they can be pressed or extruded into a core using conventional tabletting techniques as described in Remington's Pharmaceutical Sciences 18^{th} Ed. which is incorporated herein by reference.

The hydrogel forming polymer is preferably a single pharmaceutically acceptable polymeric substance such as hydroxypropyl methylcellulose. The hydroxypropyl methylcellulose of the present invention is the U.S.P. substitution type 2208 and should have an average molecular weight above 100,000, preferably above 200,000. The methyoxy content of the hydroxypropyl methylcellulose should be approximately 19-24 weight percent and the hydroxypropyl content of the hydroxypropyl methylcellulose should be approximately 7.5 to 8.5 weight percent. A suitable grade of hydroxypropyl is available from Dow Chemical CO. of Midland, Mich. Under the trade name METHOCEL KLOOM which exhibits a viscosity in a 2% aqueous solution of approximately 100,000 cps.

Other hydrogel forming polymers that can be used include carboxymethylcellulose calcium, carboxymethylcellulose sodium, guar gum, hydroxyethyl cellulose, hydroxypropyl cellulose, methyl cellulose, acrylic acid crosslinked with polyalkenyl ethers or divinyl glycol, sodium alginate and poly(ethylene oxide) (POLYOX™).

The preferred filler (or diluent) is lactose monohydrate (spray dried). Other commonly used fillers or diluents are starch, dextrose, sucrose, hydroxypropyl cellulose, microcrystalline cellulose and the like.

A fumed colloidal silicon dioxide such as CAB-O-SIL®, M5 may be used as the glidant.

A tablet lubricant may be added to the compressed core matrix. Examples of suitable tablet lubricants include magnesium stearate or glyceryl mono-stearate. EASTMAN® 600P is a commercially available material that may also be used as a lubricant. In a preferred embodiment the lubricants are passed separately through a mesh screen and then blended with a mixture of expectorant, decongestant, hydrogel forming polymer, filler and glidant which has been passed through a Comil.

Depending upon the ingredients selected for the matrix, a pre-compression step may be needed prior to actual compression in order to obtain a better tablet yield. A typical pre-compression force should be between 1 to 7 kp preferably 2 to 5 kp. The pre-compression can reduce premature capping around the edge. In order to prevent the compressed tablet cores from sticking to the tooling, the hardness of the cores should be greater than 18 kp, preferably 20-24 kp.

Once the matrix tablet core has been compressed and formed into a tablet it may optionally be seal coated. A seal coat is a coating or layer that its soluble or rapidly disintegrating in water and does not materially affect the release of the active ingredients from the tablet core. The most common seal coatings comprise low molecular weight hydroxypropyl methylcellulose or polyvinyl alcohol. Some typical seal coats are described in U.S. Pat. No. 4,786,505; 6,099,859 and 5,314,697, which are incorporated herein by reference.

An immediate release coating, preferably guaifenesin, can be coated directly onto the matrix core or applied over the sealed coated matrix core. The immediate release coating comprises an active ingredients(s), preferably an expectorant and a film forming material or binder and optionally other conventional additives such as lubricants, fillers and antiadherents.

The immediate release coating may be applied by any conventional technique such as pan coating or spray coating. In the preferred embodiment the immediate release coating is applied by spraying an aqueous solution or suspension over a pan containing the matrix core tablets. After the immediate release coating is applied to the matrix core the twelve to twenty four hour matrix tablet will comprise:

TABLE 2

| INGREDIENTS | PREFERRED | MOST PREFERRED |
|---|---|---|
| Compressed matrix core | 40-100% | 75-95% |
| Immediate release expectorant | 1-20% | 2-15% |
| Binder | 0-20% | 0.5-10% |
| Glidant | 0-10% | 0.1-5% |
| Lubricant | 0-10% | 0.1-5% |

The above percentages are based upon the total weight of the twelve to twenty four hour drug formulation or matrix tablet.

The film forming material or binder employed in the immediate release coating is preferably a water soluble or rapidly dispersing material such as a low molecular weight hydroxypropyl methylcellulose or povidone.

The matrix tablet core with or without an immediate release coating(s) can be coated with a polishing agent such as candellila wax or a color coating by any of the conventional coating techniques described in Remington's Pharmaceutical Sciences 18th Ed which is incorporated herein by reference.

Controlled Release Coated Tablet Core

If the tablet core is a controlled release coated tablet core, it should comprise a unitary, homogenous or single layer tablet core that is coated with a controlled release coating. The unitary core comprises a blend of the following materials:

TABLE 3

| Materials | Preferred | Most Preferred |
|---|---|---|
| Expectorant | 10-30% | 15-25% |
| Decongestant | 1-25% | 3-15% |
| Filler | 65-90% | 75-85% |
| Glidant | 0-2% | 0.1-1% |
| Lubricant | 0-2% | 0.1-1% |

The above percentages are based upon the total weight of the unitary core.

In forming the unitary tablet core the expectorant, decongestant and any excipients are mixed together using standard techniques known in the art. The blend is then compressed into tablets using techniques commonly used in the art.

The filler should be a water soluble or rapidly dispersible inert material such as those described above.

The preferred glidant is colloidal silicon dioxide. Other glidants commonly used in the art may be used as well.

The preferred lubricant is magnesium stearate. Other examples of suitable tablet lubricants include calcium stearate, stearic acid, hydrogenated vegetable oils, polyethylene glycol, talc, glycerol behenate and glycerol monostearate.

Once the unitary tablet core is formed, it may optionally be seal coated prior to the application of the controlled release coating.

The controlled release coating of one embodiment of the present invention comprises the following materials:

TABLE 4

| Materials | Preferred | Most Preferred |
|---|---|---|
| pH sensitive polymer | 15-60% | 25-45% |
| Plasticizer | 0-15% | 3-10% |
| Anti-adherent | 30-75% | 40-65% |

The weight percentages are based on the total weight of the controlled release coating.

The controlled release coating is applied by conventional coating techniques, such as pan coating or fluid bed coating using solutions or suspensions of polymers in water or suitable organic solvents. The controlled release coating should be applied so that the pharmaceutical active ingredient present in the core or tablet is released only after the dosage form has passed through the stomach. To insure that the pharmaceutically active ingredient present in the core is not released until the dosage form has left the stomach, the controlled release coating should be designed to dissolve at a pH greater than 4.5, preferably greater than 5.5 and most preferably greater than a pH of 6.

Preferably, the pH dependent polymer employed in the controlled release coating is selected from the group consisting of zein, methacrylic acid copolymers, cellulose acetate phthalate, hydroxypropyl methylcellulose phthalate, hydroxypropyl methylcellulose acetate succinate, cellulose acetate trimellitate, polyvinyl acetate phthalate or mixtures thereof. A mixture of zein and methacrylic acid copolymer is most preferred. The methacrylic acid copolymer is selected from the group of pH dependent coating polymers, preferably Eudragit S, and most preferably Eudragit S100. The preferred concentration of zein is 10-40%, most preferably 15-30% of the total weight of the controlled release coating. The preferred concentration of Eudragit S100 is 0-30%, most preferably 8-18% of the total weight of the controlled release coating.

The controlled release coating may also preferably comprise plasticizers. Plasticizers which may be used include any of those known to those skilled in the art, including but not limited to acetyltributyl citrate, triacetin, acetylated monoglyceride, rape oil, olive oil, sesame oil, acetyltriethyl citrate, glycerin sorbitol, diethyloxalate, diethylmalate, diethylfumerate, dibutylsuccinate, diethylmalonate, dioctylphthalate, dibutylphthalate, dibutylsebacate, triethyl citrate, tributylcitrate, glyceroltributyrate, polyethylene glycol, propylene glycol and mixtures thereof. The preferred plasticizer is acetyl tributyl citrate in an amount ranging form 0 to about 15%, and most preferably at 3-10% based on the total weight of the controlled release coating.

The controlled release coating may further preferably include an anti-adherent such as those selected from the group consisting of talc, colloidal silicon dioxide, magnesium stearate, magnesium silicate, glycerol monostearates, calcium stearate or steric acid. The preferred anti-adherent is talc in an amount ranging from about 30-75%, and most preferably 40-65% based on the total weight of the controlled release coating.

In another embodiment the controlled release coating may comprise:
(i) a pH dependent polymer;
(ii) a water insoluble polymer; and
(iii) a lubricant or dusting agent.

The ratio of the pH dependent polymer to the water insoluble polymer should be about 1:1 to 2:1, preferably 1.25:1 to 1.75:1 and most preferably about 1.5:1. These preferred ratios are based upon the weight of the pH dependent polymer to the weight of the water insoluble polymer.

Suitable pH dependent polymers are described above.

Suitable water insoluble polymers are polymers that are permeable to stomach and/or intestinal fluid and comprise polymers such as ethylcellulose, cellulose acetate, cellulose propionate, cellulose acetate propionate, cellulose acetate butyrate, cellulose acetate phthalate, cellulose triacetate, polymethyl methacrylate, polyethyl methacrylate, polybutyl methacrylate, polyisobutyl methacrylate, polyhexyl methacrylate, poly isodecyl methacrylate, polylauryl methacrylate, polyphenyl methacrylate, polymethyl acrylate, polyisopropyl acrylate, polyisobutyl acrylate, polyoctadecyl acrylate, polyethylene, polypropylene, polyethylene oxide, polyethylene terephthalate, polyvinyl isobutyl ether, polyvinyl acetate, polyvinyl chloride, polyurethane or mixtures thereof. In the preferred embodiment of the present invention, the water insoluble polymer is ethylcellulose.

In this embodiment of the present invention the controlled release coating applied to the unitary core will have the following composition:

| | Preferred | Most Preferred |
|---|---|---|
| COATING: | | |
| PH dependent polymer | 20-70% | 25-45% |
| Water insoluble polymer | 15-40% | 20-35% |
| lubricant | 25-75% | 30-60%. |

The percentages listed in the above table are based on the total weight of the coating.

Generally, the coating layer will comprise from about 1% to about 20% and preferably about 1.5% to about 15% based on the total weight of the core and coating and can be applied using conventional coating techniques such as pan coating or fluidized bed coating.

Once the controlled release coating has been applied to the unitary tablet core it may be seal coated or an immediate release layer(s) or active ingredient(s) may be applied to the controlled release coated unitary tablet core. In the present invention, the immediate release coating will comprise the expectorant, a binder and optionally conventional processing aids such as a plasticizer, a surfactant and an anti-adherent. A decongestant may be incorporated into the expectorant immediate release coating or a separate decongestant immediate release coating may be applied onto or below the immediate release expectorant coating.

The binder employed in the immediate release coating(s) is preferably a pharmaceutically acceptable water-soluble polymer or rapidly dispersing material. The binder can be any type of binding agent commonly known in the art such as sucrose, polyvinyl pyrrolidone, hydroxyethyl cellulose, hydroxypropyl cellulose, OPADRY® Clear and low molecular weight hydroxypropyl methylcellulose (HPMC). In a preferred embodiment of the present invention, the binding agent is a water-soluble polymer such as hydroxypropyl cellulose.

Osmotic Tablet

If the tablet core is an osmotic tablet, the core of the osmotic tablet can be prepared with or without a gelling or swelling polymer and should comprise a core comprising an expectorant, a decongestant and at least one pharmaceutically acceptable excipient. The osmotic core is then coated with a semipermeable membrane.

In one embodiment of the present invention the osmotic core includes the expectorant, the decongestant, a binding agent and an absorption enhancer. The osmotic core is preferably coated with a polymeric coating to form a semi-permeable membrane around the tablet and drilled to create one passageway on each side of the membrane. Preferably the osmotic tablet core of the present invention will comprise the following ingredients:

TABLE 5

| INGREDIENTS | Preferred | Most Preferred |
|---|---|---|
| Core | | |
| Active Ingredients | 50-98% | 75-95% |
| Binder | 0.1-40% | 3-15% |
| Absorption Enhancer | 0-20% | 2-10% |
| Lubricant | 0-5% | 0.5-1% |
| Coating | | |
| Semi-Permeable Polymer | 50-99% | 75-95% |
| Flux Enhancer | 0-40% | 2-20% |
| Plasticizer | 0-25% | 2-15% |

The binding agents used in the osmotic core are any conventionally known pharmaceutically acceptable binders such as polyvinyl pyrrolidone, hydroxypropyl cellulose, hydroxyethyl cellulose, hydroxypropyl methylcellulose, ethylcellulose, polymethacrylate, polyvinylalcohol, waxes and the like. Mixtures of the aforementioned binding agents may also be used. The preferred binding agents are water soluble materials such as polyvinyl pyrrolidone having an average molecular weight of 25,000 to 3,000,000. The binding agent may comprise approximately about 0% to about 40% of the total weight of the osmotic core and preferably about 3% to about 15% of the total weight of the osmotic core.

The osmotic core may also optionally comprise an absorption enhancer. The absorption enhancer can be any type of absorption enhancer commonly known in the art such as a fatty acid, a surfactant, a chelating agent, a bile salt or mixtures thereof. Examples of some preferred absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis(β-aminoethyl ether)-N,N,N,N-tetraacetic acid (EGTA). The osmotic core may comprise approximately 0% to about 20% of the absorption enhancer based on the total weight of the osmotic core and most preferably about 2% to about 10% of the total weight of the osmotic core.

In one embodiment of the present invention, which does not employ a gelling or swelling polymer, the core of the present invention is preferably formed by granulating the expectorant and decongestant with a binding agent and compressing the granules with the addition of a lubricant and absorption enhancer into a tablet. The osmotic core may also be formed by dry granulating the osmotic core ingredients by passing them through a roller compactor and compressing the granules with the addition of a lubricant into tablets. Direct compression may also be employed for tabletting. Other commonly known granulation procedures are known in the art. Additionally, other excipients such as lubricants, pigments or dyes may also be employed in the formulation of the subject invention.

The term gelling or swelling polymer refers to polymers that gel, swell or expand in the presence of water or biological fluids. Representative examples of gelling or swelling polymers are high molecular weight hydroxpropyl methylcellulose (such as METHOCEL® K100M, which is commercially available from Dow Chemical) and high molecular weight polyethylene oxides (such as POLYOX WSR 301, WSR 303 or WSR COAGULANT). Other gelling or swelling polymers are described in U.S. Pat. No. 4,522,625 (which is incorporated herein by reference).

The osmotic core can optionally be seal coated prior to the application of a membrane, preferably a semipermeable polymeric coating membrane. The semipermeable membrane is permeable to the passage of an external fluids such as water or aqueous biological fluids and is impermeable to the passage of the active ingredients in the osmotic core. Materials that are useful in forming the semipermeable membrane are ethylcellulose, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate and cellulose acetate butyrate. Other suitable polymers are described in U.S. Pat. Nos. 3,845,770; 3,916,899; 4,008,719; 4,036,228 and 4,612,008 (which are incorporated herein by reference). The most preferred semipermeable membrane material is cellulose acetate comprising an acetyl content of 39.3% to 40.3%, and is commercially available from Eastman Fine Chemicals.

In an alternative embodiment, the semipermeable membrane can include one of the above-described polymers and a flux-enhancing agent. The flux enhancing agent can increase the volume of fluid imbibed into the core to enable the dosage form to dispense substantially all of the active ingredients drug through the passageway and/or the porous membrane. The flux-enhancing agent can be a water-soluble material or an enteric material. Examples of the preferred materials that are useful as flux enhancers are sodium chloride, potassium chloride, sucrose, sorbitol, mannitol, polyethylene glycol (PEG), propylene glycol, hydroxypropyl cellulose, hydroxypropyl methycellulose, hydroxypropyl methycellulose phthalate, cellulose acetate phthalate, polyvinyl alcohols, methacrylic acid copolymers, poloxamers (such as LUTROL F68, LUTROL F127, LUTROL F108, which are commercially available from BASF) and mixtures thereof. A preferred flux-enhancer is PEG 400.

The flux enhancing agent comprises approximately 0% to about 40% of the total weight of the membrane coating, most preferably about 2% to about 20% of the total weight of the membrane coating. The flux enhancing agent dissolves or leaches from the semipermeable membrane to form paths in the semipermeable membrane which enables fluid to enter the osmotic core and dissolve the active ingredient.

The semipermeable membrane may also be formed using a commonly known excipient such as a plasticizer. Some commonly known plasticizers include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate and the like. Depending on the particular plasticizer, amounts from about 0% to about 25%, and preferably about 2% to about 15% of the plasticizer can be used based upon the total weight of the membrane coating.

Generally, the membrane coating around the core will comprise from about 1% to about 5% and preferably about 2% to about 3% based upon the total weight of the core and coating.

In a preferred embodiment, the membrane coating surrounding the core further comprises a passageway that will allow for controlled release of the drug from the core. As used herein the term passageway includes an aperture, orifice, bore, hole, weakened area or an erodible element such as a gelatin plug that erodes to form an osmotic passageway for the release of the active ingredients from the dosage form. Passageways used in accordance with the subject invention are well known and are described in U.S. Pat. Nos. 3,845,770; 3,916,899; 4,034,758; 4,077,407; 4,783,337 and 5,071,607.

An immediate release coating(s) may be applied to the semipermeable membrane. The immediate release coatings are described above and may be applied by, but would not be limited to, the processes selected from the group consisting of drug layering, lamination, dry compression, deposition and printing. In a preferred embodiment, a seal coat is applied to the semi-permeable membrane before the immediate release layer is applied.

The formulation prepared in accordance with the present invention will preferably exhibit the following expectorant/decongestant dissolution release rate when tested in 900 ml of simulated intestinal fluid using a United States Pharmacopoeia Type II apparatus at 75 rpm and 37° C.

TABLE 6

| | |
|---|---|
| EXPECTORANT | DECONGESTANT |

| | | Amount | | Amount |
|---|---|---|---|---|
| | Amount | Dissolved | Amount | Dissolved |
| | Dissolved | (Most | Dissolved | (Most |
| Time (hrs) | (Preferred) | Preferred) | (Preferred) | Preferred) |
| 1 | 0-30% | 10-25% | 0-30% | 5-25% |
| 2 | 5-40% | 15-35% | 5-40% | 10-35% |
| 4 | 15-55% | 20-50% | 10-55% | 20-50% |
| 8 | 30-75% | 35-70% | NLT 60% | NLT 65% |
| 12 | NLT 65% | NLT 70% | NLT 70% | NLT 80% |

NLT = Not Less Than

The present invention will be described in detail by reference to the following prophetic Examples.

### EXAMPLE I

An oral dosage formulation in accordance with the present invention is prepared by: (I) forming an immediate release tablet core comprising 800 mg of guaifenesin, 90 mg of pseudoephedrine HCl, lactose monohydrate, colloidal silicon dioxide and magnesium stearate; (II) coating the immediate release tablet core with a controlled release coating comprising Eudragit S 100, Zein, acetyl tributyl citrate and talc; (III) applying an immediate release expectorant and decongestant coating to the controlled release coating comprising 400 mg of guaifenesin, 30 mg of pseudoephedrine, hydroxypropyl methycellulose, polyethylene glycol and talc; (IV) seal coating the immediate release coating with OPADRY® White YS-1-7003; and (V) applying a polishing coat to the seal coated product comprising Candelilla Wax.

### EXAMPLE II

An expectorant/decongestant tablet is prepared as described in Example I wherein the core comprises 400 mg of guaifenesin and 30 mg of pseudoephedrine HCl and the immediate release coating comprises 200 mg of guaifenesin and 30 mg of pseudoephedrine HCl.

### EXAMPLE III

An expectorant/decongestant tablet is prepared as described in Example I wherein the core comprises 535 mg of guaifenesin and 50 mg of pseudoephedrine HCl and the immediate release coating comprises 265 mg of guaifenesin and 30 mg of pseudoephedrine HCl.

### EXAMPLE IV

An oral dosage formulation in accordance with the present invention is prepared by: (I) forming an immediate release tablet core comprising 800 mg of guaifenesin, 90 mg of pseudoephedrine HCl, lactose monohydrate, colloidal silicon dioxide and magnesium stearate; (II) coating the immediate release tablet core with a controlled release coating comprising Eudragit S 100, Zein, acetyl tributyl citrate and talc; (III) applying an immediate release expectorant coating to the controlled release coating comprising 400 mg of guaifenesin, hydroxypropyl methycellulose, polyethylene glycol and talc; (IV) seal coating the immediate release expectorant coating with OPADRY® White YS-1-7003; (V) applying an immediate release decongestant coating to the seal coat comprising 30 mg of pseudoephedrine, hydroxypropyl methycellulose, polyethylene glycol and talc; (VI) seal coating the immediate release decongestant coating with OPADRY® White YS-1-7003; and (VII) applying a polishing coat to the seal coated product comprising Candelilla Wax.

### EXAMPLE V

An expectorant/decongestant tablet is prepared as described in Example IV wherein the core comprises 400 mg of guaifenesin and 30 mg of pseudoephedrine HCl and the immediate release coatings comprises 200 mg of guaifenesin and 30 mg of pseudoephedrine HCl.

### EXAMPLE VI

An expectorant/decongestant tablet is prepared as described in Example IV wherein the core comprises 535 mg of guaifenesin and 50 mg of pseudoephedrine HCl and the immediate release coating comprises 265 mg of guaifenesin and 30 mg of pseudoephedrine HCl.

### EXAMPLE VII

An oral dosage formulation in accordance with the present invention is prepared by: (I) forming an immediate release tablet core comprising 800 mg of guaifenesin, 20 mg of phenylephedrine HCl, lactose monohydrate, colloidal silicon dioxide and magnesium stearate; (II) coating the immediate release tablet core with a controlled release coating comprising Eudragit S 100, Zein, acetyl tributyl citrate and talc; (III) applying an immediate release expectorant and decongestant coating to the controlled release coating comprising 400 mg of guaifenesin, 10 mg of phenylephrine HCl, hydroxypropyl methycellulose, polyethylene glycol and talc; (IV) seal coating the immediate release coating with OPADRY® White YS-1-7003; and (V) applying a polishing coat to the seal coated product comprising Candelilla Wax.

### EXAMPLE VIII

An expectorant/decongestant tablet is prepared as described in Example VII wherein the core comprises 400 mg of guaifenesin and 10 mg of phenylepherine HCl and the immediate release coating comprises 200 mg of guaifenesin and 5 mg of phenylephrine HCl.

### EXAMPLE IX

An expectorant/decongestant tablet is prepared as described in Example VII wherein the core comprises 535 mg of guaifenesin and 15 mg of phenylephrine HCl and the immediate release coating comprises 265 mg of guaifenesin and 5 mg of phenylephrine HCl.

### EXAMPLE X

An oral dosage formulation in accordance with the present invention is prepared by: (I) forming an immediate release tablet core comprising 800 mg of guaifenesin, 20 mg of phenylephrine HCl, lactose monohydrate, colloidal silicon dioxide and magnesium stearate; (II) coating the immediate release tablet core with a controlled release coating comprising Eudragit S 100, Zein, acetyl tributyl citrate and talc; (III) applying an immediate release expectorant coating to the controlled release coating comprising 400 mg of guaifenesin, hydroxypropyl methycellulose, polyethylene glycol and talc; (IV) seal coating the immediate release expectorant coating with OPADRY® White YS-1-7003; (V) applying an immediate release decongestant coating to the seal coat comprising 30 mg of phenylephrine HCl, hydroxypropyl methycellulose, polyethylene glycol and talc; (VI) seal coating the immediate release decongestant coating with OPADRY® White YS-1-7003; and (VII) applying a polishing coat to the seal coated product comprising Candelilla Wax.

### EXAMPLE XI

An expectorant/decongestant tablet is prepared as described in Example X wherein the core comprises 400 mg of guaifenesin and 10 mg of phenylephrine HCl and the immediate release coatings comprises 200 mg of guaifenesin and 5 mg of phenylephrine HCl.

### EXAMPLE XII

An expectorant/decongestant tablet is prepared as described in Example X wherein the core comprises 535 mg of guaifenesin and 15 mg of phenylephrine HCl and the immediate release coating comprises 265 mg of guaifenesin and 5 mg of phenylephrine HCl.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. An oral pharmaceutical tablet formulation comprising:
(A) A compressed core comprising:
(i) an expectorant or pharmaceutically acceptable salt thereof;
(ii) a decongestant or pharmaceutically acceptable salt thereof;
(iii) optionally a filler;
(B) a controlled release coating on said compressed core;
(C) at least one coating applied to said controlled release coating that allows for the immediate release of a therapeutic amount of an expectorant and/or a decongestant.

2. The pharmaceutical formulation defined in claim 1 wherein coating (C) comprises: I) an expectorant immediate release coating comprising: (a) an expectorant and (b) a water soluble or rapidly dispersible binder and II) a decongestant immediate release coating comprising: (a) an decongestant, (b) a water soluble or rapidly dispersible binder, and (c) a surfactant.

3. The pharmaceutical formulation defined in claim 2, further comprising a seal coat between the expectorant immediate release coating and the decongestant immediate release coating.

4. The pharmaceutical formulation defined in claim 1, wherein the expectorant is guaifenesin.

5. The pharmaceutical formulation defined in claim 1, wherein the decongestant is pseudoephedrine or phenylephrine.

6. The pharmaceutical formulation defined in claim 1, wherein the decongestant is a pseudoephedrine salt selected from the group consisting of sulfate, sodium, phosphate, or hydrochloride.

7. The pharmaceutical formulation defined in claim 1, wherein the decongestant is a phenylephrine salt selected from the group consisting of sulfate, sodium, phosphate, or hydrochloride.

8. The pharmaceutical formulation defined in claim 1, wherein the controlled release coating comprises a pH dependent polymer selected from the group consisting of zein, methacrylic acid copolymers, cellulose acetate phthalate, hydroxypropyl methylcellulose phthalate, hydroxypropyl methylcellulose acetate succinate, cellulose acetate trimellitate, polyvinyl acetate phthalate or mixtures thereof.

9. The pharmaceutical formulation defined in claim 8, wherein the pH dependent polymer is a combination of zein and methacrylic acid copolymer.

10. The pharmaceutical formulation as defined in claim 1, wherein the delayed release coating or immediate release coatings further comprises a plasticizer.

11. The pharmaceutical formulation according to claim 1 comprising 1200 mg/dose of guaifenesin and 120 mg/dose of pseudoephedrine hydrochloride.

12. The pharmaceutical formulation according to claim 1 comprising 800 mg/dose of guaifenesin.

13. The pharmaceutical formulation according to claim 1 comprising 600 mg/dose of guaifenesin and 60 mg/dose of pseudoephedrine hydrochloride.

14. The pharmaceutical formulation according to claim 1 comprising 800 mg/dose of guaifenesin and 20 mg/dose of phenylephrine hydrochloride.

15. The pharmaceutical formulation according to claim 1 comprising 600 mg/dose of guaifenesin and 15 mg/dose of phenylephrine hydrochloride.

16. A controlled release pharmaceutical formulation consisting essentially of:
(A) a compressed matrix core comprising:
(i) an expectorant;
(ii) a decongestant;
(iii) a hydrogel forming polymer;
(iv) 0 to 20 weight percent based upon the total weight of the matrix core of a filler;
(v) 0 to 5 weight percent based on the total weight of the matrix core of a glidant;
(vi) 0 to 10 weight percent based on the total weight of the matrix core of one or more lubricants;
(B) an immediate release coating on said compressed matrix core which comprising:
(i) an expectorant;
(ii) a pharmaceutically acceptable binder which allows for immediate release of the expectorant; and
(C) optionally a polishing agent or color coating that coats the immediate release coating.

17. The pharmaceutical formulation as defined in claim 16 wherein the hydrogel forming polymer is hydroxypropyl methylcellulose with an average molecular weight of 180,000 to 220,000.

18. The pharmaceutical formulation according to claim 16 comprising 1200 mg/dose of guaifenesin and 120 mg/dose of pseudoephedrine hydrochloride.

19. The pharmaceutical formulation according to claim 16 comprising 600 mg/dose of guaifenesin and 60 mg/dose of pseudoephedrine hydrochloride.

20. The pharmaceutical formulation according to claim 16 comprising 800 mg/dose of guaifenesin and 80 mg/dose of pseudoephedrine hydrochloride.

21. The pharmaceutical formulation according to claim 16 comprising 1200 mg/dose of guaifenesin and 30 mg/dose of phenylephrine hydrochloride.

22. The pharmaceutical formulation according to claim 16 comprising 800 mg/dose of guaifenesin and 20 mg/dose of phenylephrine hydrochloride.

23. The pharmaceutical formulation according to claim 16 comprising 600 mg/dose of guaifenesin and 15 mg/dose of phenylephrine hydrochloride.

24. An osmotic dosage form comprising:
(a) a homogeneous compressed core comprising:
(i) an expectorant;
(ii) a decongestant;
(ii) a binder;
(iii) optionally an absorption enhancer;
(iv) optionally a lubricant; and
(b) a membrane coating which completely covers said core tablet which comprises a mixture of a:
(i) a semi-permeable polymer;
(ii) optionally a flux enhancer; and
(iii) optionally a plasticizer.

25. The osmotic dosage form according to claim 24 comprising 1200 mg/dose of guaifenesin and 120 mg/dose of pseudoephedrine hydrochloride.

26. The osmotic dosage form according to claim 24 comprising 600 mg/dose of guaifenesin and 60 mg/dose of pseudoephedrine hydrochloride.

27. The osmotic dosage form according to claim 24 comprising 800 mg/dose of guaifenesin and 80 mg/dose of pseudoephedrine hydrochloride.

28. The osmotic dosage form according to claim 24 comprising 1200 mg/dose of guaifenesin and 30 mg/dose of phenylephrine hydrochloride.

29. The osmotic dosage form according to claim 24 comprising 800 mg/dose of guaifenesin and 20 mg/dose of phenylephrine hydrochloride.

30. The osmotic dosage form according to claim 24 comprising 600 mg/dose of guaifenesin and 15 mg/dose of phenylephrine hydrochloride.
